(19) United States
(12) Patent Application Publication
(10) Pub. No.: US 2005/0282910 A1
(43) Pub. Date: Dec. 22, 2005

(54) Methods of application of chemical compounds having therapeutic activities in treating cancers

(76) Inventor: Xun Hu, Hangzhou (CN)

Correspondence Address: CHAN LAW GROUP LC, 1055 W. 7TH ST,, SUITE 1880, LOS ANGELES, CA, 90017, US

(21) Appl. No.: 11/173,567

(22) Filed: Jul. 1, 2005

Related U.S. Application Data
(63) Continuation-in-part of application No. 10/998,025, filed on Nov. 24, 2004.

(30) Foreign Application Priority Data
Nov. 28, 2003 (CN) 200310108996X
Jun. 11, 2004 (CN) 2004100596073

(51) Int. Cl. A61K 31/415; A61K 31/135

(52) U.S. Cl. 514/656

(57) ABSTRACT

Methods of application of a class of compounds with a pharmacophore of dibenzocyclooctadiene in the preparation of anticancer medications, and particularly for the preparation of medications for the treatment of P-glycoprotein-mediated multidrug resistant (MDR) cancer and non-P-glycoprotein-mediated MDR cancer, such as multidrug resistant associated protein MRP1-mediated cancer. Methods of increasing the efficacy of anticancer agents are further disclosed. The class of compounds are of a potency to effectively reverse MDR cancer by inhibiting the drug transport activity of an ABC drug transporter, increase the intracellular accumulation of an anticancer agent in MDR cancer cells, enhance apoptosis of cancer cells induced by an anticancer agent, and directly kill cancer cells. The aforementioned methods of application of the present disclosure provide much potential for the treatment of cancer. Mass production of medications incorporating these chemical compounds will treat a significant number of patients affected by the condition of MDR cancer.

### RELATED APPLICATIONS

This application is a continuation-in-part of U.S. Ser. No. 10/998,025, filed Nov. 24, 2004, which claims the priority of Application Nos. 200310108996X and 2004100596073, filed in China on Nov. 28, 2003 and Jun. 11, 2004, respectively, pursuant to 35 U.S.C. § 119, and is incorporated herein by reference in its entirety.

### BACKGROUND OF THE INVENTION

1. Field of the Invention

This disclosure relates to a novel application of chemical compounds (Table 1) derived from Schisandra chinensis (Turcz.) Baill fruit, Schizandra sphenanthera Rehd. et Wils, and Schisandra chinensis Baill, and more specifically to the application of these compounds in the treatment of multidrug resistant (MDR) cancer. This disclosure further relates to the application of the compounds (Table 1) in increasing the efficacy of anticancer drugs.

2. Description of the Prior Art

Cancer is the leading cause of death. Chemotherapy is one of the primary ways to treat cancer. However, a major problem with chemotherapy is the ability of cancer cells to develop resistance to the cytotoxic effects of anticancer drugs during treatment. Previous studies have shown that cancer cells have the ability to become simultaneously resistant to several chemotherapeutic drugs having unrelated chemical structures and mechanisms of action. This phenomenon is commonly referred to as multidrug resistance (MDR). It has been reported that a clinically relevant and scientifically documented mechanism for MDR in cancer cells is associated with the expression of P-glycoprotein.

P-glycoprotein, an ATP binding cassette (ABC) drug transporter having a molecular weight of 170 kD, is a transmembrane protein that universally transports intracellular drugs out of the cell by catalyzing the hydrolysis of ATP. Current research tends to show that the overexpression of P-glycoprotein causes the rapid efflux of intracellular drugs, resulting in a decreased accumulation of anticancer drugs within MDR cancer cells.

Overexpression of ATP binding cassette (ABC) drug transporters, such as P-glycoprotein, multidrug resistant associated protein (MRP1), and mitoxantrone-resistance gene (MXR), in cancer cells is the most frequent cause for MDR cancer (Gottesman M M et al., Nature Medicine 2:48-58(2002)). The ABC drug transporters unilaterally pump the intracellular anticancer drug out of cells such that the drug concentration in the cancer cells is kept at a sublethal level by which cancer cells circumvent an effective attack by the anticancer drug. The expression of these drug transporters confers cancer cells with resistance toward a wide spectrum of anticancer drugs, including, but not limited to, vinca alkaloids, anthracyclines, epipodophyllotoxins, and taxans.

Expression of the ABC drug transporters referenced above accounts for clinical intrinsic and acquired MDR as reviewed by Gottesman M M et al., Nature Medicine 2:48-58(2002). Previous scientific studies have shown that a significant percentage of colorectal, kidney, adrennocortical, hepatocellular, and breast cancers, and acute myeloid leukemia at diagnosis demonstrated expression of P-glycoprotein. Similarly, a high percentage of lung cancers demonstrated overexpression of MRP1. Cancers sensitive to chemotherapy initially were frequently observed to become drug resistant due to the drug-induced expression of ABC drug transporters. A decrease in the efficacies of anticancer drugs and poor prognosis were correlated with the expression of these ABC drug transporters.

One of the ways to overcome MDR cancer is to inhibit the drug-pump activity of ABC drug transporters by use of chemical inhibitors. The inhibition of ABC drug transporters results in the increase of anticancer drug concentrations within MDR cells and restores the sensitivity of MDR cells to anticancer drugs.

As MDR reversal agents are presently lacking in clinical cancer therapy, there is a strong need to develop highly effective chemical inhibitors to clinically applicable drugs.

One of the earlier identified potent P-glycoprotein inhibitors is verapamil. However, verapamil causes severe side effects such as cardiovascular toxicity that hinder its clinical application.

It is therefore desirable to provide compounds that are useful for treating MDR cancer cells, particularly as potent ABC drug transporter inhibitors for clinically applicable drugs.

### SUMMARY OF THE INVENTION

The present disclosure concerns the discovery that a class of chemical compounds (Table 1) with a pharmacophore of dibenzocyclooctadiene (FIG. 1), derived from Schisandra chinensis (Turcz.) Baill fruit, may be useful in reversing MDR cancer by inhibiting the drug transport activity of an ABC drug transporter, increasing the intracellular accumulation of an anticancer drug in MDR cancer cells, enhancing apoptosis of cancer cells induced by an anticancer agent, and killing cancer cells. More specifically, the disclosure may be useful in treating or preventing P-glycoprotein-mediated MDR cancer and non-P-glycoprotein-mediated MDR cancer, such as MRP1-mediated MDR cancer and BCRP-mediated MDR cancer.

Schisandrin B, a compound extracted from the Chinese Schisandra chinensis (Turcz.) Baill and Schisandra sphenanthera Rehd. et Wils. plant, was previously reported to have antioxidant properties and the ability to protect against chemical-induced liver damage. The chemical structure of Schisandrin B, a derivative of dibenzocyclooctadiene, is as follows: 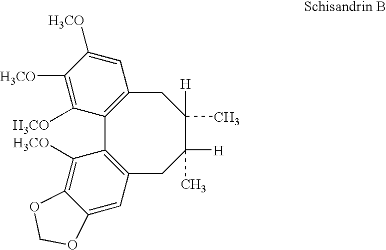
The role of Schisandrin B and the compounds set forth in Table 1 as anticancer agents has not been previously reported.

Some advantages of the application of the compound, Schisandrin B, as an anticancer agent in medications is the compound's (1) potential in clinical applications as manifested by its potency in reversing MDR cancer by inhibiting P-glycoprotein; and (2) potential as a chemotherapeutic agent against cancer as manifested by its low toxicity to normal human cells but relatively high toxicity to cancer cells. The class of compounds set forth in Table 1 shares the same pharmacophore, dibenzocyclooctadiene, as Schisandrin B, and, accordingly, possesses activities similar to that of Schisandrin B.

The present disclosure reveals that the compounds of Table 1 having the same pharmacophore of dibenzocyclooctadiene may all have the activities to reverse MDR cancer, inhibit ABC drug transporters associated with MDR cancer, enhance the anticancer activities of anticancer drugs, and directly kill cancer cells.

This disclosure provides methods of application of the class of compounds set forth in Table 1, which are of low toxicity but have strong potency in reversing MDR cancer, for use in the preparation of anticancer medications. While some compounds have stronger potencies than others within the class of compounds (Table 1), chemical modifications may be conducted within this class of known compounds by one skilled in the art in order to obtain more potent compounds for purposes of this disclosure. This disclosure further provides methods of increasing the efficacy of an anticancer agent.

The embodiments described herein particularly demonstrate that the compounds of Table 1 are of high potency in reversing MDR cancer. In comparison with verapamil, which is of high cardiovascular toxicity, the compounds of Table 1 are of very low toxicity and very safe. Because of their desirable physiological properties, the compounds of Table 1, similar to Schisandrin B, have significant potential in clinical applications, such as the preparation of anticancer medications for the treatment of cancer.

In a first aspect, the present disclosure provides a method of application of the compounds in Table 1 or an optical isomer, diasteromer, enantiomer, a pharmaceutically-accepted salt, or an analog thereof in reversing MDR cancer that comprises preparing a medication, which comprises at least one of the compounds selected from the group consisting of Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J. Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproylbinankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclootadiene], binankadsurin A, benzoylbinankadsurine A, isovaleroylbinankadsurin A, angeloylbinankadsurin A, and isobutyroylbinankadsurin A; or an optical isomer, diasteromer, enantiomer, a pharmaceutically-accepted salt, or an analog thereof. The preparation of the medication further comprises incorporating at least one anticancer chemotherapeutic agent and a pharmaceutically accepted carrier.

The chemotherapeutic agents are selected from the group consisting of doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof. The medication is formulated for administration in the form of a capsule, caplet, tablet, pill, suspension, or liquid. The medication includes at least one MDR reversal agent.

The preparation of the medication further comprises increasing intracellular accumulation of an anticancer drug in MDR cancer cells, inhibiting drug-pump activity of at least one ABC drug transporter, such as P-glycoprotein (P-gp, or ABCB1—ATB binding cassette, subclass B, member 1), multidrug resistant associated protein 1 (MRP1, or ABCC1—ATB binding cassette, subclass C, member 1), multidrug resistant associated protein 2 (MRP2, or ABCC2—ATB binding cassette, subclass C, member 2), multidrug resistant associated protein 3 (MRP3, or ABCC3—ATB binding cassette, subclass C, member 3), multidrug resistant associated protein 4 (MRP4, or ABCC4—ATB binding cassette, subclass C, member 4), multidrug resistant associated protein 5 (MRP5, or ABCC5—ATB binding cassette, subclass C, member 5), breast cancer resistant protein (BCRP, or ABCG2—ATB binding cassette, subclass G, member 2, or MRX—mitoxantrone resistance gene, or ABCB—placental ABC protein), enhancing apoptosis of cancer cells induced by an anticancer agent, or killing cancer cells.

In other aspects, the present disclosure provides methods of increasing efficacies of an anticancer agent comprising co-administering to a subject suffering from MDR cancer a dose of the anticancer agent, wherein the anticancer agent is a substrate of an ABC drug transporter, such as P-glycoprotein, MRP1, MRP2, MRP3, MRP4, MRP5, BCRP, and a dose of a compound in Table 1 or an optical isomer, diasteromer, enantiomer, a pharmaceutically-accepted salt, or an analog thereof. The co-administration to a subject suffering from MDR cancer further comprises administering an optional dose of physiologically acceptable adjuvants, diluents, excipients, or carries. The dose of the anticancer agent is a therapeutic or subtherapeutic dose.

The anticancer agent is selected from the group consisting of doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

The dose of the compound or the analog thereof further comprises reducing efflux of the anticancer agent from a cancer cell, increasing intracellular concentration of the anticancer agent in a cancer cell, or inhibiting a host drug transporter.

In a further aspect, the present disclosure provides a method of decreasing toxicity associated with treating a subject with an anticancer agent comprising co-administering to the subject having a cancer a dose of the anticancer agent, and a dose of a compound of Table 1 or an optical isomer, diasteromer, enantiomer, a pharmaceutically-accepted salt, or an analog thereof. The co-administration to a patient having a cancer further comprises administering an optional dose of physiologically acceptable adjuvants, diluents, excipients, or carries.

In still another aspect, the present disclosure provides a method of enhancing the anticancer activity of an anticancer agent against a cancer cell comprising co-administering to a subject suffering from MDR cancer a dose of the anticancer agent, and a dose of a compound of Table 1 or an optical isomer, diasteromer, enantiomer, a pharmaceutically-accepted salt, or an analog thereof. The co-administration to a subject suffering from MDR cancer further comprises administering an optional dose of physiologically acceptable adjuvants, diluents, excipients, or carries.

In a further aspect, the present disclosure provides a method of increasing the efficacy of an anticancer agent comprising co-administering to a subject suffering from MDR cancer a dose of the anticancer agent, wherein the anticancer agent is a substrate of an ABC drug transporter, and a dose of ethanol extracts of Schisandra chinensis (Turcz.) Baill fruit.

In still a further aspect, the present disclosure provides a method of treating a subject suffering from a cancer comprising administering a therapeutic dose of a compound of Table 1 or an optical isomer, diasteromer, enantiomer, a pharmaceutically-accepted salt, or an analog thereof.

In yet another aspect, the present disclosure provides a method of increasing oral bioavailability of a drug comprising co-administering to a subject a dose of the drug, wherein the drug is a substrate of an ABC drug transporter and a dose of a compound of Table 1.

In a further aspect, the present disclosure provides a method of optimizing pharmacotherapy of a drug comprising co-administering to a subject a dose of the drug, wherein the drug is a substrate of an ABC drug transporter and a dose of a compound of Table 1.

These and other features and advantages of this disclosure will become further apparent from the detailed description and accompanying figures that follow.

### BRIEF DESCRIPTION OF THE DRAWINGS

FIG. 1 is a chemical formula of dibenzocyclooctadiene.

FIG. 2 is a graph illustrating the reversal of resistance of MDR cancer cell K562/adr to doxorubicin in the presence or absence of ethanol extracts of S. chinesis (Turcz.) Baill fruit.

FIG. 3 is a graph illustrating the reversal of resistance of MDR cancer cell K562/adr to daunorubicin in the presence or absence of Schisandrin A, Schisandrol A, or Schisantherin A.

FIG. 4 is a graph illustrating the effects of Schisandrin A, Schisandrol A, or Schisantherin A on the accumulation of daunorubicin in MDR cancer cell K562/adr.

FIG. 5 is a graph illustrating the effects of Schisandrin A, Schisandrol A, or Schisantherin A on the accumulation of daunorubicin in MDR cancer cell MCF7/adr.

FIG. 6 is a graph illustrating the effects of Schisandrin B on the accumulation of daunorubicin in MDR cancer cell HL60/adr.

FIG. 7 is a graph illustrating the effects of Schisandrin A, Schisandrol A, or Schisantherin A on the accumulation of daunorubicin in MDR cancer cell HL60/adr.

### DETAILED DESCRIPTION OF THE INVENTION

The terms and abbreviations used in the Detailed Description set forth herein have their normal meanings unless otherwise specified. For example, “° C.” refers to degrees Celsius; “g” refers to gram or grams; “ml” refers to milliliter or milliliters; “μg” refers to microgram or micrograms; “ng” refers to nanogram and nanograms; “nm” refers to nanometer and nanometers; and “IC₅₀” refers to the inhibitory concentration of a drug that causes 50% inhibition of the cells.

The present disclosure is directed towards the treatment of various types of cancer or diseases by inhibiting ABC drug transporters associated with MDR cancer, increasing intracellular accumulation of anticancer agents in MDR cells, enhancing the anticancer activities of anticancer agents, and killing cancer cells. ABC drug transporter P-glycoprotein is expressed with a high incidence in, but not limited to, colorectal, kidney, adrenocortical, breast, ovary, or hepatocellular cancers; sarcomas; and leukemia. MRP1 is expressed with a high incidence in, but not limited to, lung and breast cancers, and leukemia. Other ABC drug transporters such as BCRP can be overexpressed in leukemia and breast cancer. The class of compounds (Table 1) disclosed herein and derived from Schisandra chinensis (Turcz.) Baill fruit have the potential to treat other types of diseases in addition to cancer.

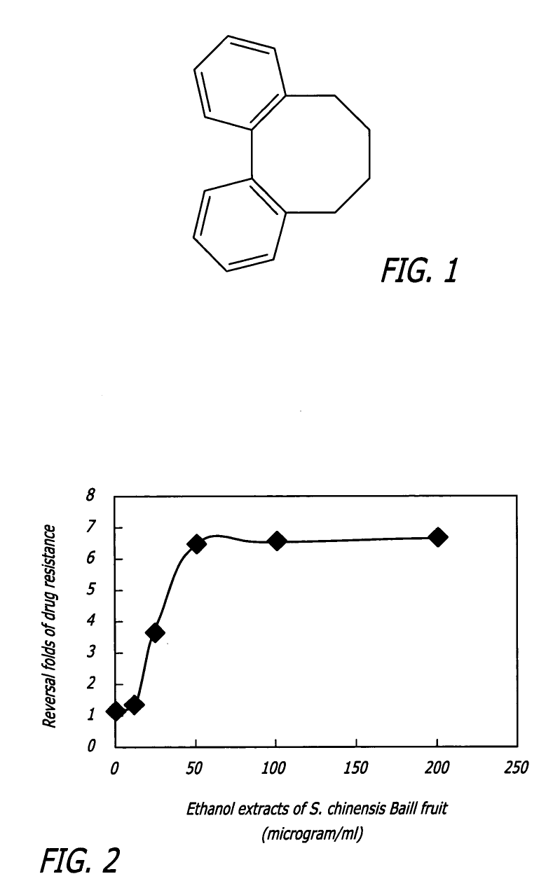

As previously disclosed in U.S. Ser. No. 10/998,025, filed on Nov. 24, 2004 and incorporated herein by reference in its entirety, Schisandrin B, a Schisandra chinensis (Turcz.) Baill fruit derived compound with the pharmacophore of dibenzocyclooctadiene, was shown to have potency in reducing MDR cancer, inhibiting ABC drug transporters associated with MDR cancer, increasing intracellular accumulation of anticancer agents, enhancing the anticancer activities of anticancer agents, and killing cancer cells. In accordance with the present disclosure, it is claimed that compounds with the pharmacophore of dibenzocyclooctadiene (FIG. 1) may possess similar functions as Schisandrin B although some compounds may be more potent than others in the class. Table 1 sets forth the list of compounds derived from natural sources such as Schisandra chinensis (Turcz.) Baill fruit, Schizandra sphenanthera Rehd. et Wils, and Schisandra chinensis Baill, or from chemical synthesis and contemplated for use in accordance with the present disclosure. The compounds are not limited to those listed in Table 1 but may also include optical isomers, diasteromers, enantiomers, pharmaceutically-accepted salts or analogs thereof.

TABLE 1
Compounds derived from Schisandra chinensis (Turcz.) Baill fruit

| Compound | Formula | Name |
|---|---|---|
| 1 | 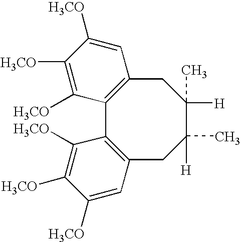 | Schisandrin A |
| 2 | 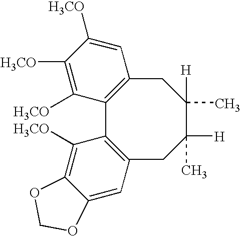 | Schisandrin B |
| 3 | 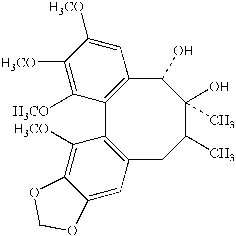 | Schisandrin A |
| 4 | 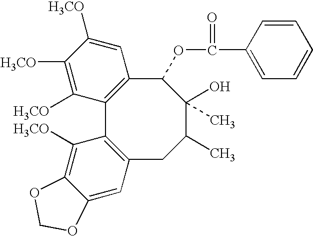 | Schisantherin A |
| 5 | 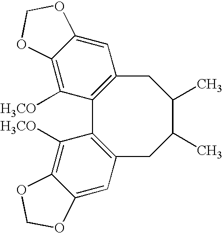 | Schisandrin C |
| 6 | 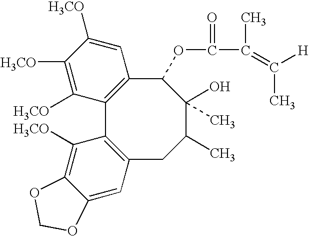 | Schisantherin B |
| 7 | 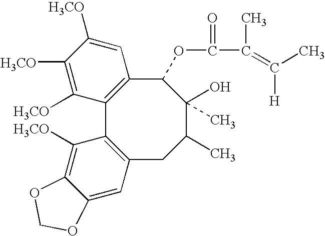 | Schisantherin C |
| 8 | 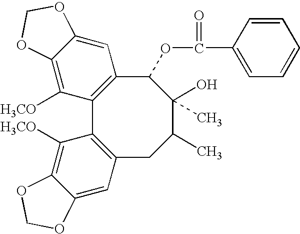 | Schisantherin D |
| 9 | 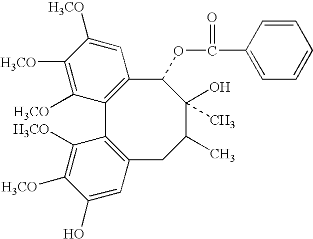 | Schisantherin E |
| 10 | 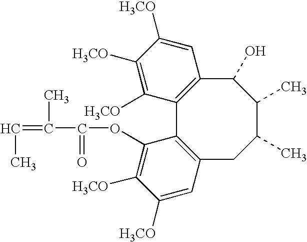 | Schisantherin F |
| 11 | 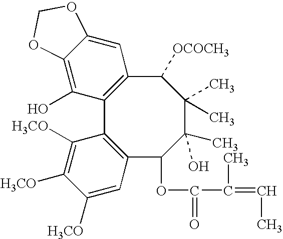 | Schisantherin G |
| 12 | 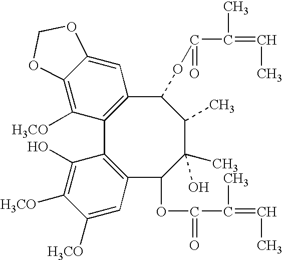 | Schisantherin H |
| 13 | 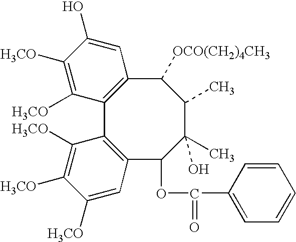 | Schisantherin I |
| 14 | 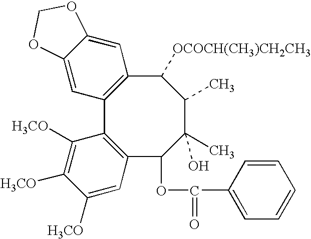 | Schisantherin J |
| 15 | 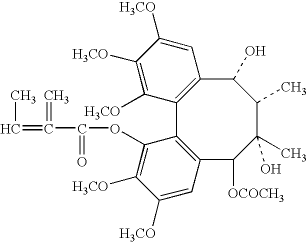 | Schisantherin K |
| 16-18 | 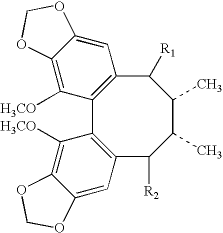 | 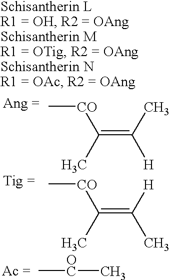 |
| 19 | 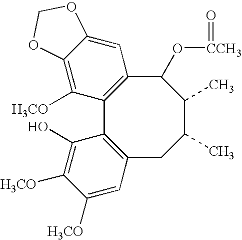 | Schisantherin O |
| 20 | 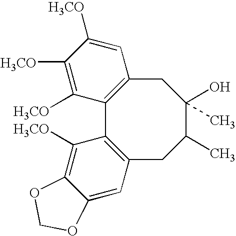 | Schisandrol B Schisantherinol B |
| 21 | 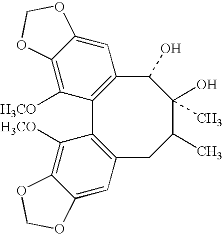 | Schisandrol D Schisantherinol D |
| 22 | 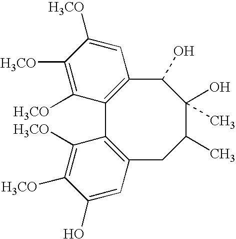 | Schisandrol E Schisantherinol E |
| 23 | 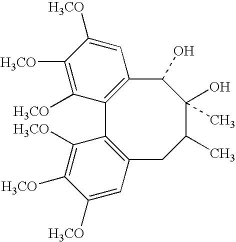 | Methylschisandrol E Methylschisantherinol E |
| 24-25 | 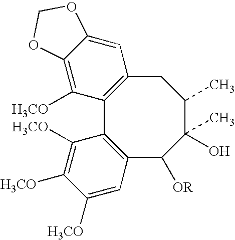 | 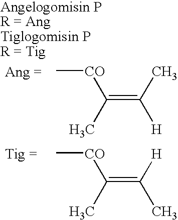 |
| 26 | 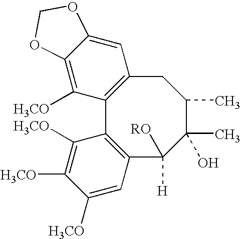 | Gomisin A |
| 27-28 | 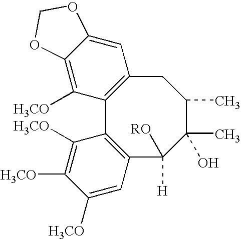 | 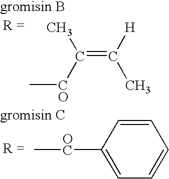 |
| 29 | 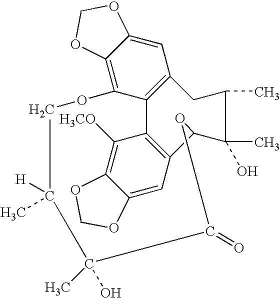 | Gomisin D |
| 30 | 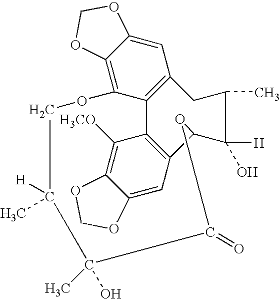 | Gomisin E |
| 31 | 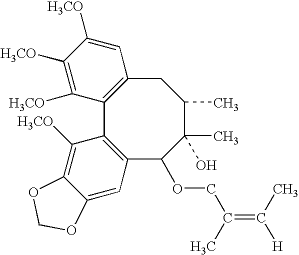 | Gomisin F |
| 32 | 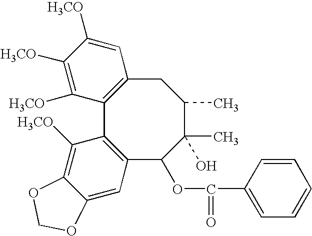 | Gomisin G |
| 33 | 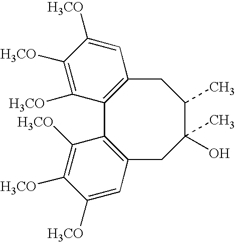 | Gomisin H |
| 34 | 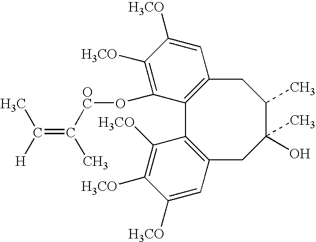 | Angeloylgomisin H |
| 35 | 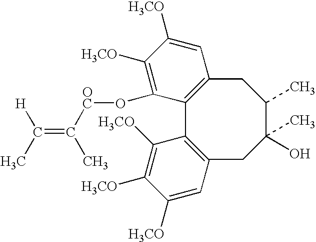 | Tigloylgomisin H |
| 36 | 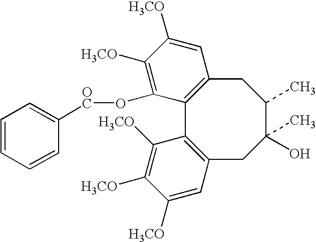 | Benzoylgomisin H |
| 37 | 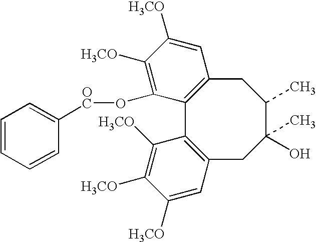 | Gomisin J |
| 38 | 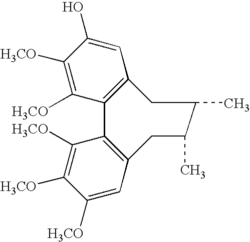 | Gomisin K1 |
| 39 | 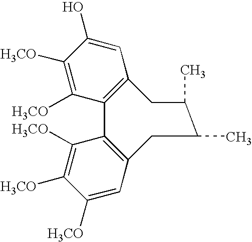 | Gomisin K2 |
| 40 | 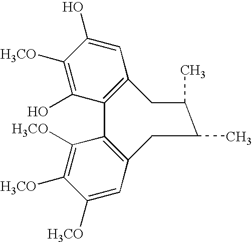 | Gomisin K3 |
| 41 | 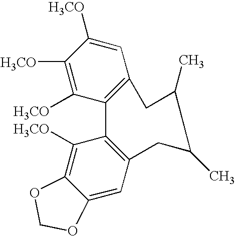 | Gomisin M1 |
| 42 | 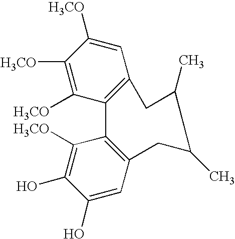 | Gomisin M2 |
| 43 | 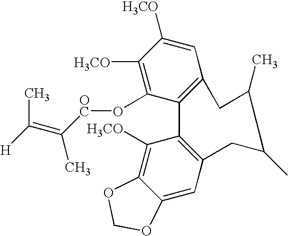 | Angeloylgomisin M1 |
| 44 | 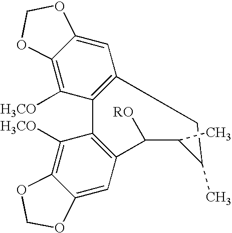 | 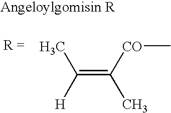 |
| 45 | 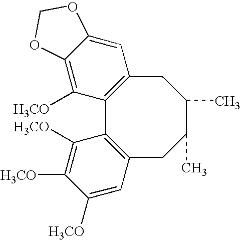 | Gomisin N |
| 46 | 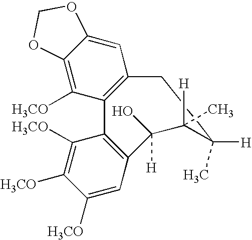 | Gomisin O |
| 47 | 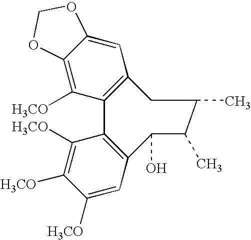 | Epigomisin O |
| 48 | 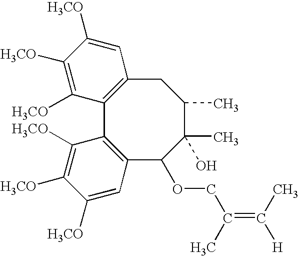 | Gomisin Q |
| 49 | 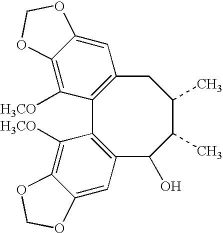 | Gomisin R |
| 50 | 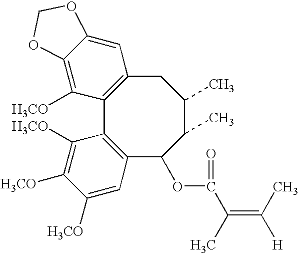 | Angelogomisin O |
| 51 | 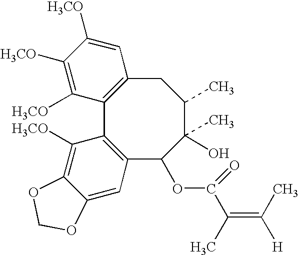 | Angeloisogomisin O |
| 52 | 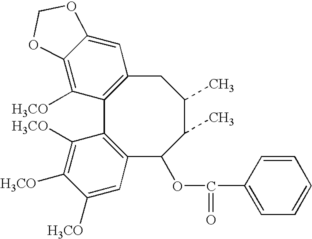 | Benzoylgomisin O |
| 53 | 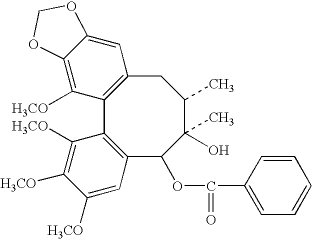 | Benzoylgomisin P |
| 54 | 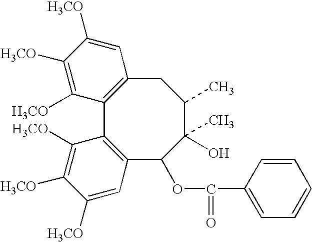 | Benzoylgomisin Q |
| 55 | 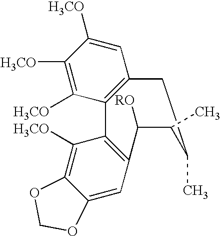 | 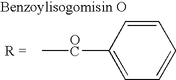 |
| 56-60 | 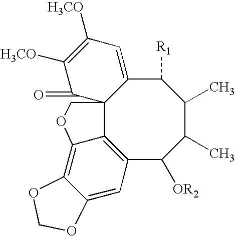 | 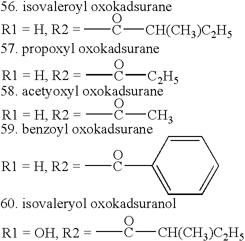 |
| 61-63 | 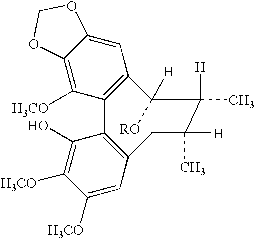 | 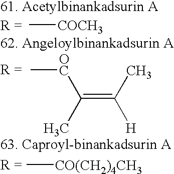 |
| 64 | 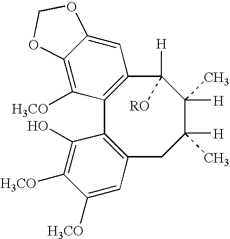 | kadsurin R = —CXOCH₃ |
| 65 | 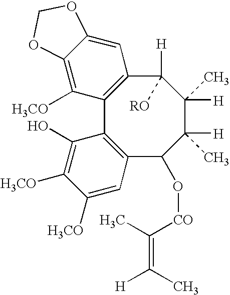 | kadsurin R = —COCH₃ |
| 66 | 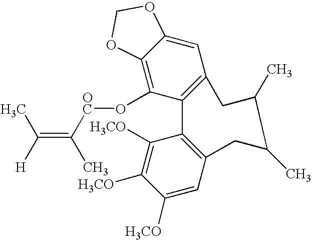 | kadasutherin |
| 67 | 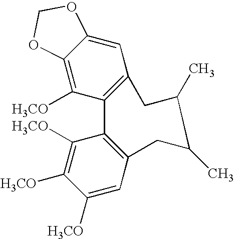 | isokadsuranin |
| 68 | 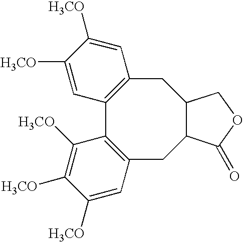 | neoisostegane |
| 69 | 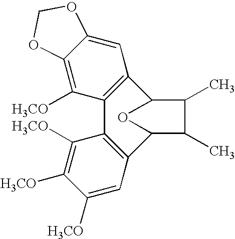 | neokadsuranin |
| 70 | 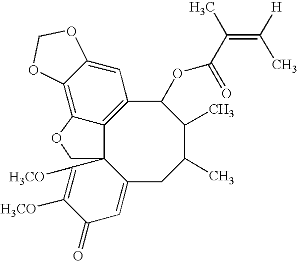 | Interiorin |
| 71 | 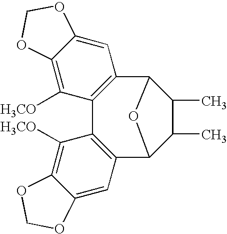 | 5,8-epoxy-6,7-dimethyl 2′,3′,2″,3″- dimethylenedioxy-4′,1″- dimethoxy-1,2:3,4-dibenzo- 1,3-cyclootadiene |
| 72-76 | 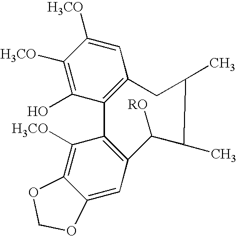 | 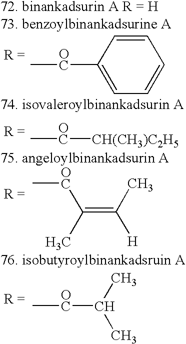 |

As used herein, the term “optical isomer” is equivalent to the term “stereoisomer,” which are isomers that have the same atom connectivity but differ only in their orientation in space. Stereoisomers include geometrical isomers, diasteromers, and enantiomers. “Enantiomers” are stereoisomers that are non-superimposeable mirror images of one another. “Diasteromers” are stereoisomers that are not mirror images of one another. “Geometrical isomers (cis-trans)” are stereoisomers about a double bond.

The term “pharmaceutically accepted salt” as used herein refers to the formation of a salt from the reaction of a compound of Table 1 with an inorganic or organic acid or base. Such salt is known as an acid addition or base addition salt, respectively.

The term “acid addition salt” refers to a salt of a compound of Table 1 prepared by reaction of a compound of Table 1 with a mineral or organic acid. The compounds of the present disclosure can react with any number of inorganic and organic acids to form pharmaceutical acid addition salts. The pharmaceutical acid addition salts of the disclosure may be formed by reacting the compound of Table 1 with an equimolar or excess amount of acid. The reactants are typically combined in a mutual solvent such as diethylether, tetrahydrofuran, methanol, ethanol, isopropanol, or benzene. The salts precipitate out of solution generally within about one hour to about several days and can be isolated by filtration or other conventional methods.

Examples of acids typically used in the formation of acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenylsulfonic acid, carbonic acid, succinic acid, citric acid, benzoic acid, acetic acid, and the like. Examples of such pharmaceutically accepted salts are sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, β-hydroxybutyrate, glycollate, tartrate, methanesulfonate, propanesulfonate, naphthalene-1-sulfonate, naphthalene-2-sulfonate, mandelate and the like.

The term “base addition salt” refers to a salt of a compound of Table 1 prepared by reaction of a compound of Table 1 with a mineral or organic base. Because some of the compounds of Table 1 may be acidic in nature, the compounds may accordingly react with a variety of inorganic and organic bases to form pharmaceutical base addition salts. Examples of base addition salts are ammonium, lithium, potassium, sodium, calcium, magnesium, methylamino, diethylamino, ethylene diamino, cyclohexylamino, and ethanolamino salts, and the like of a compound of Table 1.

“Analogs” are compounds with the pharmacophore of dibenzocyclooctadiene.

In consideration of the beneficial effect on the reversal of MDR cancer produced by the application of the compounds of Table 1 or optical isomers, diasteromers, enantiomers, pharmaceutically-accepted salts, or analogs thereof in anticancer medications, these compounds may also be useful not only for therapeutic treatment after the onset of MDR, but also for prevention of MDR in patients about to undergo chemotherapy for the first time.

Anticancer medications prepared according to this disclosure may be formulated in various forms for administration, including, but not limited to, tablets, caplets, capsules, pills, suspensions, liquids and the like. As used herein, “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired.

Acceptable solid carriers may include one or more substances that may also act as flavoring agents, lubricants, solubilizers, suspending agents, fillers, glidants, compression aids, binders or tablet-disintegrating agents or an encapsulating material. In tablets, the active ingredient may be mixed with a carrier having the necessary compression properties in suitable proportions and compacted in the shape and size desired. Acceptable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Any suitable liquid carriers may be used in preparing solutions, suspensions, emulsions, syrups and elixirs according to the present disclosure. The select compound of Table 1 or an optical isomer, diasteromer, enantiomer, a pharmaceutically-accepted salt, or an analog thereof, may be dissolved or suspended in any pharmaceutically acceptable liquid carrier such as water, one or more organic solvents, mixtures of both or pharmaceutically acceptable oils or fat. The liquid carrier may also include other suitable pharmaceutical additives such as solubilizers, emulsifiers, buffers, preservatives, viscosity regulators, stabilizers or osmoregulators. Exemplary liquid carriers for oral and parenteral administration include water (especially containing additives as above, e.g., cellulose derivative and sodium carboxymethyl cellulose solution), alcohols (e.g., monohydric alcohols and polyhydric alcohols) and their derivatives, and oils (e.g., fractionated coconut oil and arachis oil). For parenteral administration the pharmaceutically acceptable carrier may also be an oily ester such as ethyl oleate and isopropyl myristate. Sterile liquid carriers may also be used in sterile liquid form compositions for parenteral administration.

Any suitable liquid pharmaceutical compositions that are sterile solutions or suspensions may be utilized by, for example, intramuscular or subcutaneous injection. Sterile solutions can also be administered intravenously. Oral administration may be either in the form of a solid or liquid composition.

As used herein, the term “dose” refers to a specified quantity of a therapeutic agent, such as a drug or medicine, prescribed to be taken at one time or at stated intervals.

The anticancer agent and the compounds of Table 1 or an optical isomer, diasteromer, enantiomer, a pharmaceutically-accepted salt, or an analog thereof, according to the present disclosure may be administered using any dose (e.g., therapeutic or subtherapeutic) and any route of administration effective for treating MDR cancer cells. The administration of a therapeutically effective dose is generally desirable. A therapeutically effective dose refers to a nontoxic but sufficient amount of the MDR reversal agent to provide the desired effect against the MDR cells. The exact amount will vary from subject to subject, depending on such factors as the species, age, general medical condition of the subject, the particular MDR reversal agent, its mode of administration and the like.

Although treatment using any of the compounds of Table 1 or an optical isomer, diasteromer, enantiomer, a pharmaceutically-accepted salt, or an analog thereof, according to the present disclosure described herein, may be administered to any subject susceptible to the development of MDR, methods of treatment according to the present disclosure are intended particularly for the treatment of cancer in humans.

The compounds of Table 1 according to the present disclosure are extracted from the Chinese Schisandra chinensis (Turcz.) Baill and Schisandra sphenanthera Rehd. et Wils. plant according to various procedures well known to those of ordinary skill in the art.

In a first embodiment according to the present disclosure, a method of application of the compounds of Table 1 or an optical isomer, diasteromer, enantiomer, pharmaceutically-accepted salt, or analog thereof, in reversing MDR cancer that includes preparing a medication comprising at least one of the compounds selected from the class of compounds of Table 1 or an optical isomer, diasteromer, enantiomer, pharmaceutically-accepted salt, or analog thereof, is disclosed. It is contemplated by the present disclosure that the anticancer medication prepared with the at least one compound of Table 1 or an optical isomer, diasteromer, enantiomer, pharmaceutically-accepted salt, or analog thereof, can include at least one anticancer chemotherapeutic agent that can be combined with a pharmaceutically acceptable carrier as described herein. The anticancer chemotherapeutic agent may be selected from doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, Schisandrin B, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof. The anticancer medication prepared by the present disclosure can include a compound of Table 1 or an optical isomer, diasteromer, enantiomer, pharmaceutically-accepted salt, or analog thereof, combined with other MDR reversal agents such as XR-9576, R-101933, and LY-335979 (Gottesman M M et al., Nat Rev/Cancer 2:48-58 (2001)).

In other embodiments, the present disclosure provides methods of increasing efficacies of an anticancer agent that includes co-administering to a subject suffering from MDR cancer a dose of the anticancer agent, which is a substrate of an ABC drug transporter, and a dose of a compound of Table 1 or an optical isomer, diasteromer, enantiomer, pharmaceutically-accepted salt, or an analog thereof. An optional dose of physiologically acceptable adjuvants, excipients, or carries may be administered.

The anticancer agent may be selected from doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, Schisandrin B, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

It is contemplated by the present disclosure that the ATP binding cassette (ABC) drug transporters include P-gp, MRP1, and BCRP. Additional ABC drug transporters contemplated for use in accordance with the present disclosure include MRP2, MRP3, MRP4, MRP5, and the like.

In another embodiment, the present disclosure provides a method of decreasing toxicity associated with treating a subject with an anticancer agent that includes co-administering to the subject having a cancer a dose of the anticancer agent, and a dose of a compound of Table 1 or an optical isomer, diasteromer, enantiomer, pharmaceutically-accepted salt, or an analog thereof. An optional dose of physiologically acceptable adjuvants, excipients, or carries may be administered.

In a further embodiment, the present disclosure provides a method of enhancing anticancer activity of an anticancer agent against a cancer cell that includes co-administering to the subject suffering from MDR cancer a dose of the anticancer agent, and a dose of a compound of Table 1 or an optical isomer, diasteromer, enantiomer, pharmaceutically-accepted salt, or an analog thereof. An optional dose of physiologically acceptable adjuvants, excipients, or carries may be administered.

In a still further embodiment, the present disclosure provides a method of increasing efficacy of an anticancer agent that includes co-administering to a subject suffering from MDR cancer a dose of the anticancer agent, which is a substrate of an ABC drug transporter, and a dose of ethanol extracts of Schisandra chinensis (Turcz.) Baill fruit.

In another embodiment, the present disclosure provides a method of treating a subject suffering from a cancer that includes administering a therapeutic dose of a compound of Table 1 or an optical isomer, diasteromer, enantiomer, pharmaceutically-accepted salt, or an analog thereof. An optional dose of physiologically acceptable adjuvants, excipients, or carries may be administered.

The embodiments set forth herein establish that the compounds of Table 1 are highly effective in reversing MDR cancer by inhibiting the drug-transport activity of various ABC drug transporters, such as P-glycoprotein, MRP1, MRP2, MRP3, MRP4, MRP5, and BCRP in MDR cancer cells. In particular, the compounds of Table 1 inhibit the expression of the aforementioned ABC drug transporters in MDR cancer cells. By incorporating the compounds of Table 1 into anticancer medications prepared by the methods of this disclosure, the compounds bind with the ABC drug transporter and effectively compete with anticancer agents in reversing MDR cancer. The compounds of Table 1 in accordance with the methods of the present disclosure are also effective in increasing intracellular concentration of the anticancer agent in a cancer cell. The compounds of Table 1 further have the ability to induce apoptosis or death of cancer cells.

The following embodiments are intended to illustrate and not to limit the disclosure.

To illustrate a method for reversal of P-glycoprotein-mediated drug resistance of MDR cancer cells by ethanol extracts of Schisandra chinensis (Turcz.) Baill fruit, 50 g of Schisandra chinensis (Turcz.) Baill fruit was extracted using 200 ml 95% ethanol. The extracts were concentrated and dissolved into dimethyl sulfoxide (DMSO). IC₅₀s of anticancer agents in the presence or absence of ethanol extracts of Schisandra chinensis (Turcz.) Baill fruit toward MDR cells, including K562/adr (X. Hu et al., Acta Pharmacol. Sin 15:422-426 (1994)), KBv200 (X. H. Zhang et al., Vincristine-resistant human KB cell line and mechanism of multidrug resistance, Yao Xue Xue Bao 29:246-251 (1994)), and MCF7/adr (C. R. Fairchild et al., Cancer Res. 47:5141-5148 (1987)), were determined. These cell lines were selected for use because they are characteristic of overexpression of P-glycoprotein.

Assays were carried out in triplicate against human MDR cancer cell lines K562/adr, KBv200, and MCF7/adr. MTT assays as previously described (X. Hu et al., Chemotherapy 41:296-305 (1995)) were used to determine the cytotoxicity of the anticancer agent in the presence or absence of ethanol extracts of Schisandra chinensis (Turcz.) Baill fruit. The treatment of the MDR cancer cells were incubated at 37° C. for 72 hours in a humidified CO₂ incubator. The cell number in each sample was estimated by correlating to optical density at 595 nm. The median dose value was determined from plots of median effects and was equivalent to IC₅₀. Alternatively, flow cytometry assays using FACS Calibur equipped with software Cellquest 3.1f (Becton-Dickinson, Holbrook, N.J.) were applied to count the cell numbers in each sample. The median dose value was determined from plots of median effects and was equivalent to IC₅₀.

As shown in FIG. 2, the sensitivity of K562/adr toward doxorubicin increased in the presence of the ethanol extracts of Schisandra chinensis (Turcz.) Baill fruit at concentrations ranging from 1 to 50 μg/ml. A further increase of the concentration of ethanol extracts of Schisandra chinensis (Turcz.) Baill fruit did not increase the sensitivity of K562/adr toward doxorubicin.

Table 2 is a summary of the reversal of drug resistance of K562/adr to anticancer agents by ethanol extracts of Schisandra chinensis (Turcz.) Baill fruit.

TABLE 2
Reversal of drug resistance of K562/adr by ethanol extracts of
Schisandrin chinensis (Turcz.) Baill fruit (50 μg/ml)

| | |
|---|---|
| Anticancer | IC50 (ng/ml) |

| agent | −ethanol extract | +ethanol extract | RF |
|---|---|---|---|
| Daunorubicin | 1610 ± 350 | 150 ± 19 | 10.7 |
| Taxol | 110 ± 20 | 41 ± 24 | 2.7 |
| Vincristine | 870 ± 230 | 110 ± 42 | 7.9 |

With reference to Table 2, “− ethanol extract” represents cells treated with anticancer agents in the absence of ethanol extracts of Schisandra chinensis (Turcz.) Baill fruit; “+ ethanol extract” represents cells treated with anticancer agents in the presence of ethanol extracts of Schisandra chinensis (Turcz.) Baill fruit; and “RF” represents reversal folds of drug resistance.

Table 3 is a summary of the reversal of drug resistance of KBv200 to anticancer agents by ethanol extracts of Schisandra chinensis (Turcz.) Baill fruit.

TABLE 3
Reversal of drug resistance of KBv200 by ethanol extracts of
Schisandra chinensis (Turcz.) Baill fruit (50 μg/ml)

| | |
|---|---|
| Anticancer | IC50 (ng/ml) |

| agent | −ethanol extract | +ethanol extract | RF |
|---|---|---|---|
| Daunorubicin | 20 ± 8 | 2.0 ± 0.4 | 10 |
| Vincristine | 42 ± 14 | 3.1 ± 0.5 | 13.5 |

Referring now to Table 3, “− ethanol extract” represents cells treated with anticancer agents in the absence of ethanol extracts of Schisandra chinensis (Turcz.) Baill fruit; “+ ethanol extract” represents cells treated with anticancer agents in the presence of ethanol extracts of Schisandra chinensis (Turcz.) Baill fruit; and “RF” represents reversal folds of drug resistance.

Table 4 is a summary of the reversal of drug resistance of MCF7/adr to anticancer agents by ethanol extracts of Schisandra chinensis (Turcz.) Baill fruit.

TABLE 4
Reversal of drug resistance of MCF7/adr by ethanol extracts of
Schisandra chinensis (Turcz.) Baill fruit (50 μg/ml)

| | |
|---|---|
| Anticancer | IC₅₀ (ng/ml) |

| agent | −ethanol extract | +ethanol extract | RF |
|---|---|---|---|
| Daunorubicin | 320 ± 71 | 31 ± 10 | 10.3 |
| Taxol | 73 ± 27 | 25 ± 12 | 2.9 |
| Vincristine | 490 ± 112 | 210 ± 32 | 2.3 |

With reference to Table 4, “− ethanol extract” represents cells treated with anticancer agents in the absence of ethanol extracts of Schisandra chinensis (Turcz.) Baill fruit; “+ ethanol extract” represents cells treated with anticancer agents in the presence of ethanol extracts of Schisandra chinensis (Turcz.) Baill fruit; and “RF” represents reversal folds of drug resistance.

The results of this embodiment indicate that the ethanol extracts of Schisandra chinensis (Turcz.) Baill fruit have strong activities in reversing MDR cancer. Schisandrin B, a compound of Table 1, constitutes about 0.4% of the ethanol extracts of Schisandra chinensis (Turcz.) Baill fruit extracts. In the assays of reversing drug resistance of MDR cells by the ethanol extracts of Schisandra chinensis (Turcz.) Baill fruit, the concentration (0.4×50 μg/ml=2 μg/ml) of Schisandrin B is much lower than its effective concentration (10 μg/ml). The reversal of drug resistance of MDR cancer cells by ethanol extracts of Schisandra chinensis (Turcz.) Baill fruit is contributed to the compounds of Table 1 because these compounds are the dominant compounds present in Schisandra chinensis (Turcz.) Baill fruit. Each of the compounds of Table 1 also has the core structure of dibenzocyclooctadiene and are the primary compounds in ethanol extracts (J. L. Hancke et al., Fitoterapia 70:451-471 (1999) and references therein).

To illustrate the reversal of P-glycoprotein-mediated drug resistance of MDR cancer cells by Schisandrin A, Schisandrol A, and Schisantherin A according to the present disclosure, the IC₅₀s of anticancer agents in the presence or absence of Schisandrin A, Schisandrol A, or Schisantherin A toward MDR cells were determined. Assays were carried out in triplicate against human leukemia MDR cell line K562/adr, human breast cancer MDR cell line MCF7/adr, and human epidermoid carcinoma MDR cell line KBv200. MTT assays as described previously (X. Hu et al., Chemotherapy 41:296-305 (1995)) were used to determine the cytotoxicity of each anticancer agent in the presence or absence of Schisandrin B or verapmil. The treatment of the above cells lasted for 72 hours in a humidified CO₂ incubator at 37° C. The cell number in each sample was estimated by correlating to optical density at 595 nm. The median dose value was determined from plots of median effects and was equivalent to IC₅₀. Alternatively, flow cytometric assays were applied to count the cell numbers of each sample. The median dose value was determined from plots of median effects and was equivalent to IC₅₀.

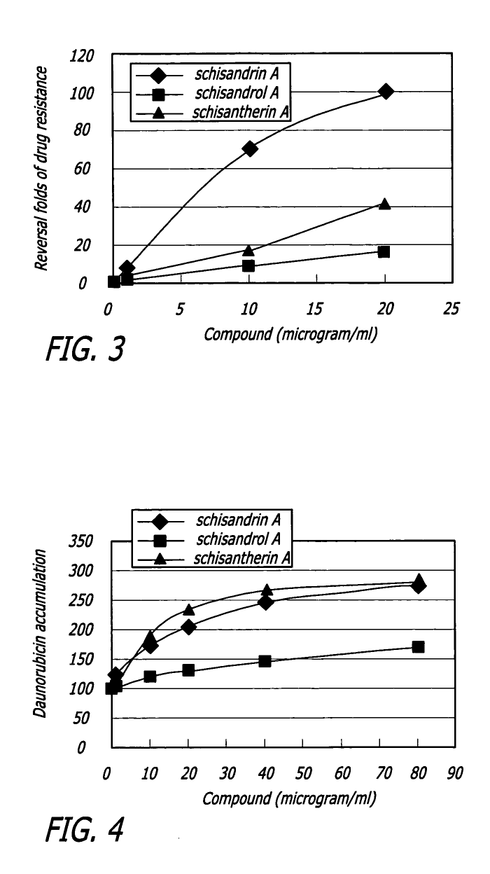

As illustrated in FIG. 3, Schisandrin A, Schisandrol A, or Schisantherin A, each has activities that increase the sensitivity of MDR cancer cell K562/adr to daunorubicin, although the potency of these compounds to reverse MDR cancer varies. In the presence of Schisandrin A at concentrations of 1, 10, and 20 μg/ml, the sensitivity of K562/adr to daunorubicin increased to about 8, 71, and 100 folds, respectively.

Table 5 is a summary of the reversal of drug resistance of K562/adr to anticancer agents by Schisantherin A.

TABLE 5
Reversal of drug resistance of K562/adr by Schisantherin A (10 μg/ml)

| | |
|---|---|
| Anticancer | IC₅₀ (ng/ml) |

| agent | −Schisantherin A | +Schisantherin A | RF |
|---|---|---|---|
| Daunorubicin | 1297 ± 293 | 71 ± 22 | 18.3 |
| Vincristine | 612 ± 158 | 7.7 ± 2.1 | 79.5 |
| Taxol | 38 ± 12 | 1.1 ± 1.0 | 34.5 |

Table 5 sets forth the results of K562/adr cells treated with anticancer agents in the absence or presence of Schisantherin A. Cells were treated with specific anticancer agents as set forth therein. The term “− Schisantherin A” represents cells treated with anticancer agents in the absence of Schisantherin A; “+ Schisantherin A” represents cells treated with anticancer agents in the presence of Schisantherin A; and “RF” represents reversal folds of drug resistance, which is determined by the IC₅₀ in the absence of Schisantherin A divided by the IC₅₀ in the presence of Schisantherin A.

Table 6 is a summary of the reversal of drug resistance of KBv200 to anticancer agents by Schisantherin A.

TABLE 6
Reversal of drug resistance of KBv200 by Schisantherin A (10 μg/ml)

| | |
|---|---|
| Anticancer | IC₅₀ (ng/ml) |

| agent | −Schisantherin A | +Schisantherin A | RF |
|---|---|---|---|
| Daunorubicin | 26.9 ± 4.3 | 3.4 ± 1.3 | 7.9 |
| Vincristine | 22.5 ± 5.6 | 3.7 ± 0.9 | 6.1 |
| Taxol | 20.2 ± 3.8 | 2.4 ± 0.7 | 8.4 |

Table 6 sets forth the results of KBv200 cells treated with anticancer agents in the absence or presence of Schisantherin A. Cells were treated with specific anticancer agents as set forth therein. The term “− Schisantherin A” represents cells treated with anticancer agents in the absence of Schisantherin A; “+ Schisantherin A” represents cells treated with anticancer agents in the presence of Schisantherin A; and “RF” represents reversal folds of drug resistance, which is determined by the IC₅₀ in the absence of Schisantherin A divided by the IC₅₀ in the presence of Schisantherin A.

Table 7 is a summary of the reversal of drug resistance of MCF7/adr to anticancer agents by Schisantherin A.

TABLE 7
Reversal of drug resistance of MCF7/adr by
Schisantherin A (10 μg/ml)

| | |
|---|---|
| Anticancer | IC₅₀ (ng/ml) |

| agent | −Schisantherin A | +Schisantherin A | RF |
|---|---|---|---|
| Daunorubicin | 812 ± 193 | 58.8 ± 21 | 13.8 |
| Vincristine | 501 ± 109 | 24.1 ± 9.2 | 20.9 |
| Taxol | 40.6 ± 20.3 | 6.3 ± 3.1 | 6.4 |

Table 7 sets forth the results of MCF7 cells treated with anticancer agents in the absence or presence of Schisantherin A. Cells were treated with specific anticancer agents as set forth therein. The term “− Schisantherin A” represents cells treated with anticancer agents in the absence of Schisantherin A; “+ Schisantherin A” represents cells treated with anticancer agents in the presence of Schisantherin A; “RF” represents reversal folds of drug resistance, which is determined by the IC₅₀ in the absence of Schisantherin A divided by the IC₅₀ in the presence of Schisantherin A.

Table 8 is a summary of the reversal of drug resistance of K562/adr to anticancer agents by Schisandrin A.

TABLE 8
Reversal of drug resistance of K562/adr by Schisandrin A (10 μg/ml)

| | |
|---|---|
| Anticancer | IC₅₀ (ng/ml) |

| agent | −Schisandrin A | +Schisandrin A | RF |
|---|---|---|---|
| Daunorubicin | 4781 ± 1382 | 67.2 ± 21.3 | 71.2 |
| Vincristine | 612 ± 176 | 7.3 ± 2.1 | 83.8 |
| Taxol | 38 ± 11 | 6.4 ± 3.2 | 5.9 |

Table 8 sets forth the results of K562/adr cells treated with anticancer agents in the absence or presence of Schisandrin A. Cells were treated with specific anticancer agents as set forth therein. The term “− Schisandrin A” represents cells treated with anticancer agents in the absence of Schisandrin A; “+ Schisandrin A” represents cells treated with anticancer agents in the presence of Schisandrin A; and “RF” represents reversal folds of drug resistance, which is determined by the IC₅₀ in the absence of Schisandrin A divided by the IC₅₀ in the presence of Schisandrin A.

Table 9 is a summary of the reversal of drug resistance of KBv200 to anticancer agents by Schisandrin A.

TABLE 9
Reversal of drug resistance of KBv200 by Schisandrin A (10 μg/ml)

| | |
|---|---|
| Anticancer | IC₅₀ (ng/ml) |

| agent | −Schisandrin A | +Schisandrin A | RF |
|---|---|---|---|
| Daunorubicin | 26.9 ± 8.1 | 2.47 ± 0.9 | 10.9 |
| Vincristine | 22.5 ± 3.2 | 2.95 ± 0.7 | 7.6 |
| Taxol | 20.2 ± 2.8 | 1.91 ± 1.2 | 10.6 |

Table 9 sets forth the results of KBv200 cells treated with anticancer agents in the absence or presence of Schisandrin A. Cells were treated with specific anticancer agents as set forth therein. The term “− Schisandrin A” represents cells treated with anticancer agents in the absence of Schisandrin A; “+ Schisandrin A” represents cells treated with anticancer agents in the presence of Schisandrin A; and “RF” represents reversal folds of drug resistance, which is determined by the IC₅₀ in the absence of Schisandrin A divided by the IC₅₀ in the presence of Schisandrin A.

Table 10 is a summary of the reversal of drug resistance of MCF7/adr to anticancer agents by Schisandrin A.

TABLE 10
Reversal of drug resistance of MCF7/adr by Schisandrin A (10 μg/ml)

| | |
|---|---|
| Anticancer | IC₅₀ (ng/ml) |

| agent | −Schisandrin A | +Schisandrin A | RF |
|---|---|---|---|
| Daunorubicin | 2556 ± 413 | 245 ± 46 | 10.4 |
| Vincristine | 501 ± 129 | 8.4 ± 2.3 | 59.6 |
| Taxol | 40.5 ± 11 | 6.1 ± 1.9 | 6.6 |

Table 10 sets forth the results of MCF7/adr cells treated with anticancer agents in the absence or presence of Schisandrin A. Cells were treated with specific anticancer agents as set forth therein. The term “− Schisandrin A” represents cells treated with anticancer agents in the absence of Schisandrin A; “+ Schisandrin A” represents cells treated with anticancer agents in the presence of Schisandrin A; and “RF” represents reversal folds of drug resistance, which is determined by the IC₅₀ in the absence of Schisandrin A divided by the IC₅₀ in the presence of Schisandrin A.

Schisandrol A showed activities reversing drug resistance of K562/adr. In the absence of Schisandrol A, the IC₅₀ of doxorubicin toward K562/adr is 875±248 ng/ml. However, in the presence of Schisandrol A (10 μg/ml), the IC₅₀ of doxorubicin toward K562/adr is 168±43 ng/ml.

In accordance with the present disclosure, the results of this embodiment set forth in Tables 5-10 above indicate that Schisandrin A, Schisandrol A, and Schisantherin A, have the activities to effectively reverse MDR cancer.

The following embodiment illustrates that the compounds of Table 1, such as Schisandrin A, Schisandrol A, and Schisantherin A, inhibit P-glycoprotein-mediated drug efflux in MDR cancer cells according to the present disclosure.

P-glycoprotein functions as a drug pump that unilaterally pumps the anticancer agents out of MDR cancer cells. Inhibition of P-glycoprotein results in an increase of the intracellular drug concentration within cancer cells. The inhibition of P-glycoprotein is assessed by analyzing the anticancer agent concentration within the test cells in the presence or absence of Schisandrin A, Schisandrol A, or Schisantherin A.

MDR cancer cells K562/adr and MCF7/adr were separately incubated in RPMI-1640 complete medium containing 2 μg/ml daunorubicin in the presence or absence of Schisandrin A, Schisandrol A, or Schisantherin A (0, 1, 10, 20, 40, and 80 μg/ml) at 37° C. Cells were collected at 60 minutes after incubation. Cells were washed twice with ice-cold phosphate buffered saline and the daunorubicin concentration within cells was measured by flow cytometry at excitation wavelength of 488 nm and emission wavelength of 533 nm using a FACS Calibur equipped with software Cellquest 3.1f (Becton-Dickinson, Holbrook, N.J.).

FIG. 4 illustrates that in the absence of Schisandrin A, Schisandrol A, or Schisantherin A, K562/adr cells retain less daunorubicin within cells than in the presence of Schisandrin A, Schisandrol A, or Schisantherin A. The accumulation of daunorubicin within cells is proportionately correlated with the increment of the concentration of Schisandrin A, Schisandrol A, or Schisantherin A.

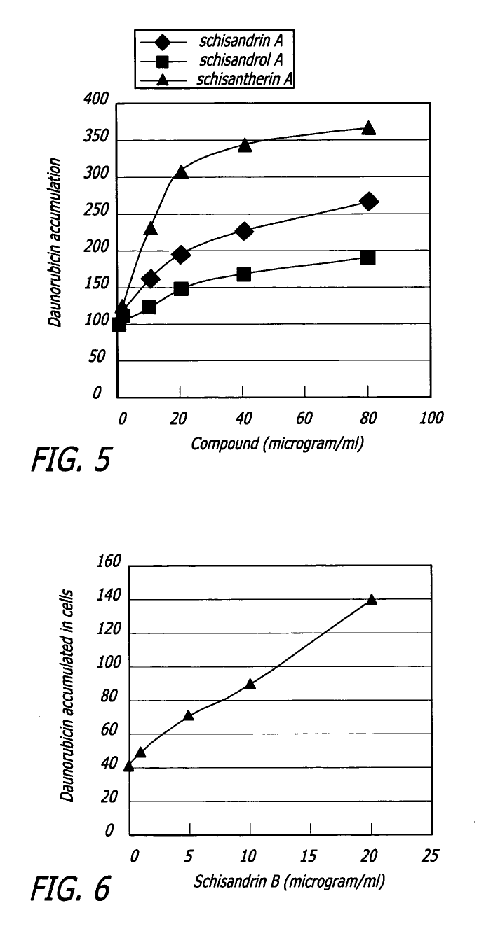

FIG. 5 illustrates that in the absence of Schisandrin A, Schisandrol A, or Schisantherin A, MCF7/adr cells retain less daunorubicin within cells than in the presence of Schisandrin A, Schisandrol A, or Schisantherin A. The accumulation of daunorubicin within cells is proportionately correlated with the increment of the concentration of Schisandrin A, Schisandrol A, or Schisantherin A.

This embodiment according to the present disclosure demonstrates that Schisandrin A, Schisandrol A, and Schisantherin A, are able to increase the anticancer agent concentration within MDR cancer cells.

To illustrate the reversal of multidrug resistant associated protein MRP1-mediated drug resistance of MDR cancer cells by Schisandrin A, Schisandrin B, Schisandrol A, and Schisantherin A according to the present disclosure, human cancer cell line HL60/adr, an MDR cell line characterized by MRP1 overexpression (W. March et al., Cancer Res. 46:4053-4057 (1986)), was selected for use. Assays were carried out in triplicate against MDR cancer cell HL60/adr. MTT assays as described previously (X. Hu et al., Chemotherapy 41:296-305 (1995)) were used to determine the cytotoxicity of each anticancer agent in the presence or absence of Schisandrin A, Schisandrin B, Schisandrol A, and Schisantherin A. The treatment of the above cells lasted for 72 hours in a humidified CO₂ incubator at 37° C. The cell number in each sample was estimated by correlating to optical density at 595 nm. The median dose value was determined from plots of median effects and was equivalent to IC₅₀. Alternatively, flow cytometric assays were applied to count the cell numbers of each sample. The median dose value was determined from plots of median effects and was equivalent to IC₅₀.

As set forth in Table 11, Schisandrin B increased the sensitivity of MDR cancer cell HL60/adr to vincristine. In the presence of Schisandrin B at incremental concentrations of 5, 10, and 20 μg/ml, the sensitivity of HL60/adr to vincristine increased about 8.4, 10.5, and >42 folds, respectively, demonstrating a dose-and-effect relationship.

TABLE 11
Reversal of vincristine resistance of HL60/adr by Schisandrin B
at different concentrations

| | |
|---|---|
| Schisandrin B | IC₅₀ (ng/ml) |

| (μg/ml) | −Schisandrin B | +Schisandrin B | RF |
|---|---|---|---|
| 5 | 42 ± 1 | 5 ± 1 | 8.4 |
| 10 | 42 ± 1 | 4 ± 2 | 10.5 |
| 20 | 42 ± 1 | <1 | >42 |

With reference to Table 11, the term “− Schisandrin B” represents cells treated with vincristine in the absence of Schisandrin B; “+ Schisandrin B” represents cells treated with vincristine in the presence of Schisandrin B; and “RF” represents reversal folds of drug resistance, which is determined by the IC₅₀ in the absence of Schisandrin B divided by IC₅₀ in the presence of Schisandrin B.

Table 12 is a summary of the reversal of drug resistance of HL60/adr to anticancer agents by Schisandrin B. Schisandrin B enhanced the drug sensitivity of HL60/adr toward the anticancer agents.

TABLE 12
Reversal of drug resistance of MDR cell HL60/adr by Schisandrin B
(10 μg/ml).

| | |
|---|---|
| Anticancer | IC₅₀ (ng/ml) |

| agent | −Schisandrin B | +Schisandrin B | RF |
|---|---|---|---|
| Daunorubicin | 460 ± 43 | 56 ± 12 | 8.3 |
| Vincristine | 48 ± 12 | <1 | >48 |
| Taxol | 3 ± | <1 | >3 |
| VP-16 | >10000 | 1490 ± 213 | >6.7 |
| Mitoxantrone | 68 | <1 | >68 |

Table 12 sets forth the results of MDR cell HL60/adr treated with anticancer agents in the absence or presence of Schisandrin B. Cells were treated with specific anticancer agents as set forth therein. The term “− Schisandrin B” represents cells treated with anticancer agents in the absence of Schisandrin B; “+ Schisandrin B” represents cells treated with anticancer agents in the presence of Schisandrin B; and “RF” represents reversal folds of drug resistance, which is determined by IC₅₀ in the absence of Schisandrin B divided by IC₅₀ in the presence of Schisandrin B.

Table 13 is a summary of the reversal of drug resistance of HL60/adr to anticancer agents by Schisandrin A. Schisandrin A enhanced the drug sensitivity of HL60/adr toward the anticancer agents.

TABLE 13
Reversal of drug resistance of MDR cell HL60/adr by Schisandrin A
(10 μg/ml).

| | |
|---|---|
| Anticancer | IC₅₀ (ng/ml) |

| agent | −Schisandrin A | +Schisandrin A | RF |
|---|---|---|---|
| Daunorubicin | 161 ± 25 | 52 ± 13 | 3.1 |
| Vincristine | 89 ± 21 | 3.3 ± 0.9 | 27.0 |
| Taxol | 32 ± 9 | 4.1 ± 1.4 | 7.8 |

Table 13 sets forth the results of HL60/adr cells treated with anticancer agents in the absence or presence of Schisandrin B or verapamil. Cells were treated with specific anticancer agents as set forth therein. The term “− Schisandrin A” represents cells treated with anticancer agents in the absence of Schisandrin A; “+ Schisandrin A” represents cells treated with anticancer agents in the presence of Schisandrin A; and “RF” represents reversal folds of drug resistance, which is determined by IC₅₀ in the absence of Schisandrin A divided by IC₅₀ in the presence of Schisandrin A.

Table 14 is a summary of the reversal of drug resistance of HL60/adr to anticancer agents by Schisantherin A. Schisantherin A enhanced the drug sensitivity of HL60/adr toward the anticancer agents.

TABLE 14
Reversal of drug resistance of MDR cell HL60/adr by Schisantherin A
(10 μg/ml).

| | |
|---|---|
| Anticancer | IC₅₀ (ng/ml) |

| agent | −Schisantherin A | +Schisantherin A | RF |
|---|---|---|---|
| Daunorubicin | 161 ± 25 | 57 ± 13 | 2.8 |
| Vincristine | 89.1 ± 21 | 3.9 ± 0.9 | 22.8 |
| Taxol | 32 ± 9 | 4.3 ± 1.4 | 7.4 |

Table 14 sets forth the results of HL60/adr cells treated with anticancer agents in the absence or presence of Schisantherin A. Cells were treated with specific anticancer agents as set forth therein. The term “− Schisantherin A” represents cells treated with anticancer agents in the absence of Schisantherin A; “+ Schisantherin A” represents cells treated with anticancer agents in the presence of Schisantherin A; and “RF” represents reversal folds of drug resistance, which is determined by IC₅₀ in the absence of Schisandrin A divided by IC₅₀ in the presence of Schisantherin A.

This embodiment according to the present disclosure demonstrates that Schisandrin A, Schisandrin B, Schisandrol A, and Schisantherin A, are able to increase the anticancer agent concentration within MDR cancer cells.

The following embodiment illustrates that Schisandrin A, Schisandrin B, Schisandrol A, and Schisantherin A, inhibit MRP1-mediated drug efflux in MDR cancer cells.

MRP1 functions as a drug pump that unilaterally pumps the anticancer agents out of MDR cancer cells. Inhibition of MRP1 results in an increase of intracellular drug concentration within cancer cells. The inhibition of MRP1 is assessed by analyzing the anticancer agent concentrations within the test cells in the presence or absence of Schisandrin A, Schisandrin B, Schisandrol A, or Schisantherin A.

MDR cancer cells HL60/adr were incubated in RPMI-1640 complete medium containing 2 μg/ml daunorubicin in the presence or absence of Schisandrin A, Schisandrin B, Schisandrol A, or Schisantherin A (0, 1, 5, 10, 20, 40, or 80 μg/ml) at 37° C. Cells were collected at 60 minutes (Schisandrin A, Schisandrol A, and Schisantherin A) or 90 minutes (Schisandrin B) after incubation. Cells were washed twice with ice-cold phosphate buffered saline and the daunorubicin concentration within cells was measured by flow cytometry at excitation wavelength of 488 nm and emission wavelength of 533 nm using FACS Calibur equipped with software Cellquest 3.1f (Becton-Dickinson, Holbrook, N.J.).

As illustrated in FIG. 6, in the absence of Schisandrin B, HL60/adr cells accumulated significantly less daunorubicin than the cells in the presence of Schisandrin B. Daunorubicin accumulation within HL60/adr cells increased proportionately with increasing concentration of Schisandrin B, demonstrating a dose and effect relationship. HL60/adr cells accumulated 4 folds more daunorubicin in the presence of Schisandrin B (20 μg/ml) than in the absence of Schisandrin B.

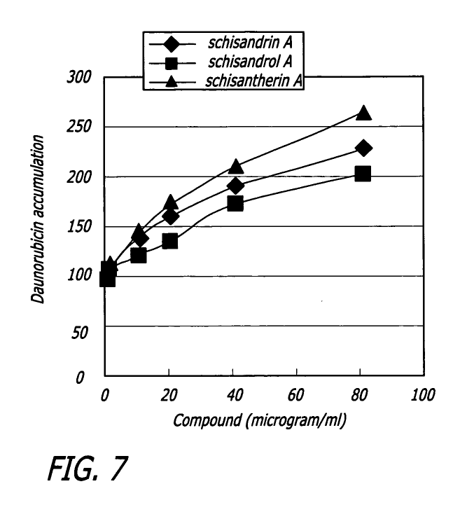

As illustrated in FIG. 7, in the absence of Schisandrin A, Schisandrol A, or Schisantherin A, HL60/adr cells accumulated significantly less daunorubicin than the cells in the presence of Schisandrin A, Schisandrol A, or Schisantherin A. Daunorubicin accumulation within HL60/adr cells increased with the increasing concentration of these compounds, demonstrating a dose and effect relationship.

To illustrate the activities of Schisandrin B or its analogs enhancing the apoptosis of cancer cells induced by an anticancer agent according to the present disclosure, human cancer cell line SMMC7721, a non-drug-resistant hepatic cancer cell line, and MCF7, a non-drug-resistant breast cancer cell line, were selected for use. Assays were carried out in triplicates against SMMC7721 or MCF7. Cells were treated with doxorubicin (0.5 μg/ml) or vincristine (0.5 μg/ml) in the absence or presence of Schisandrin B (20 μg/ml) and incubated at 37° C. for 48 hours in a humidified CO₂ incubator. Cells were collected and the apoptotic cells and necrotic cell percentages were determined using Anexin V-FITC/PI kit (Sigma, St. Louis, Mo., USA) according to manufacturer's instructions. Alternatively, the apoptotic and necrotic cell percentages were measured using DNA Plus kit (Becton-Dickinson, USA) according to the manufacturer's instructions.

Table 15 is a summary of Schisandrin B in the enhancing of apoptosis and necrosis of SMMC7721 induced by doxorubicin.

TABLE 15
Schisandrin B enhances the apoptosis of SMMC7721 induced by
doxorubicin

| | Control | Sch B alone | Dox alone | Dox + Sch B |
|---|---|---|---|---|
| Apoptotic(%) | 1.8 ± 0.2 | 2.4 ± 2.1 | 5.2 ± 2.6 | 12.1 ± 1.1 |
| Necrotic(%) | 1.4 ± 0.2 | 1.5 ± 0.9 | 16.5 ± 4.3 | 27.4 ± 0.6 |

Table 15 sets forth the results of SMMC7721 cells treated with doxorubicin in the absence or presence of Schisandrin B. The term “control” represents cells incubated in the absence of doxorubicin and Schisandrin B; “Sch B alone” represents cells incubated in the presence of schisandrin alone; “Dox alone” represents cells incubated in the presence of doxorubicin only; “Dox+Sch B” represents cells incubated in the presence of doxorubicin and Schisandrin B. The results indicate that while Schisandrin B alone caused significant increase of neither apoptotic cells nor necrotic cells, it significantly enhanced the apoptosis and necrosis of SMMC7721 cells induced by doxorubicin, indicating Schisandrin B or its analogs are of potential as a sensitizer.

Table 16 is a summary of Schisandrin B in the enhancing of apoptosis and necrosis of SMMC7721 induced by vincristine.

TABLE 16
Schisandrin B enhances the apoptosis and necrosis of SMMC7721
induced by vincristine

| | Control | Sch B alone | Vcr alone | Vcr + Sch B |
|---|---|---|---|---|
| Apoptotic + necrotic | 1.6 ± 0.3 | 2.6 ± 0.4 | 6.1 ± 0.4 | 12.4 ± 1.4 |
| (%) | | | | |

Table 16 sets forth the results of SMMC7721 cells treated with vincristine in the absence or presence of Schisandrin B. The term “control” represents cells incubated in the absence of vincrsitine and Schisandrin B; “Sch B alone” represents cells incubated in the presence of schisandrin alone; “Vcr alone” represents cells incubated in the presence of vincristine only; “Vcr+Sch B” represents cells incubated in the presence of vincristine and Schisandrin B. The results indicate that while Schisandrin B alone did not cause significant increase of apoptotic and necrotic cells, it significantly enhanced the apoptosis and necrosis of SMMC7721 induced by vincristine, indicating Schisandrin B or its analogs are of potential as a sensitizer.

Table 17 is a summary of Schisandrin B in the enhancing of apoptosis and necrosis of MCF7 induced by doxorubicin.

TABLE 17
Schisandrin B enhances the apoptosis and necrosis of MCF7 induced
by doxorubicin

| | Control | Sch B alone | Dox alone | Dox + Sch B |
|---|---|---|---|---|
| Apoptotic + necrotic | 1.1 ± 0.4 | 1.9 ± 0.1 | 7.2 ± 0.8 | 16.4 ± 0.4 |
| (%) | | | | |

Table 17 sets for the results of MCF7 cells treated with doxorubicin in the absence or presence of Schisandrin B. The term “control” represents cells incubated in the absence of doxorubicin and Schisandrin B; “Sch B alone” represents cells incubated in the presence of Schisandrin alone; “Dox alone” represents cells were incubated in the presence of doxorubicin only; “Dox+Sch B” represents cells incubated in the presence of doxorubicin and Schisandrin B. The results indicate that while Schisandrin B alone did not cause significant increase of apoptotic and necrotic cells, it significantly enhanced the apoptosis and necrosis of MCF7 induced by doxorubicin, indicating Schisandrin B or its analogs are of potential as a sensitizer.

The compounds listed in Table 1 are also of potential in improving oral bioavailability of many diverse drugs that are substrates of ABC drug transporters. The ABC drug transporters are expressed in the normal tissues and organs. For example, P-gp is mainly expressed in the epithelial cells in the body, where it localizes to the apical membrane. As consequence, transported P-gp substrates are translocated from the basolateral to the apical side of the epithelium. This can have dramatic consequences for the pharmacological behavior of the substrate drugs. As P-gp is abundant in the intestinal epithelium, it could restrict the rate at which substrate compounds present in the intestinal lumen enter the bloodstream. Many drugs are P-gp substrates, so that the oral bioavailability of these drugs is to a large extent restricted. The other ABC drug transporters expressed in intestinal tract are MRP2, MRP4, and BCRP. These ABC drug transporters play a critical role in restricting the oral bioavailability of many diverse drugs, not only anticancer drugs, but also other drugs, including antibiotics, antiviral agents, anti-gout agents, antidiarrheal agents, immunosuppressive agents, corticoids, antiemetics, cardiac glycosides, and the like (Schinkel A H & Jonker J W. Advanced Drug Delivery Review, 55:3-29, 2003). Therefore, the general pharmacotherapeutic relevance is the capability to improve the oral bioavailability of the ABC drug transporter's substrate drugs.

Increased oral bioavailability of topotecan, a BCRP substrate, was achieved by co-administration of a BCRP inhibitor GF120918 (Kruijtzer C M F, Beijnen J H, Rosing H, et al: J Clin Oncol. 20:2943-2950, 2002). Paclitaxel, a substrate of P-gp, improved dramatically for its oral bioavailability by co-administration with P-gp inhibitors PSC 833, GF120918, cyclosporine A (Schinkel A H & Jonker J W. Advanced Drug Delivery Review, 55:3-29, 2003). Accordingly, the ABC drug transporter inhibitors can generally improve oral bioavailability of the drugs that are substrates of ABC drug transporters, and hence the compounds listed in Table 1 are potent inhibitors of P-gp and other ABC drug transporters. Thus, the compounds set forth in Table 1 are of potential in the application for the preparation of medications for improving the oral bioavailability of the drugs as substrates of ABC drug transporters.

Further potentials of the compounds listed in Table 1 are for the pharmacotherapeutic optimization of substrate drugs for ATP drug transporters. Endothelial cells of the small blood capillaries in the brain are closely linked to each other by tight junctions, and cover the entire wall of these blood vessels. This structure is called blood-brain barrier. P-gp and MRP2 are abundantly expressed in the lumen membrane of the endothelial cells, which block the entrance of drugs to the brain side. Gliomas, which might position behind blood-brain barrier, is poorly accessible to most of the anticancer drugs. For other diseases of the central nervouos system, it might likewise be desirable to improve the brain parenchyma penetration of drugs. To achieve this purpose, the P-gp inhibitors, such as PSC 833 and GF120918, have been shown to improve the penetration of drugs into brain parenchyma (Schinkel A H & Jonker J W. Advanced Drug Delivery Review, 55:3-29, 2003). Similarly, P-gp and other drug transporters in the other barriers, such as blood-testis barrier, blood-nerve barrier, fetal-maternal barrier, or in the heptobiliary and renal excretion systems, could be modulated by the corresponding inhibitors for the purpose of pharmacotherapeutic optimization of drugs. Since the compounds in Table 1 are potent inhibitors of P-gp and other drug transporters, these compounds are of potential in the preparation of medications for the pharmacotherapeutic optimization of drugs related to P-gp, MRP1, MRP2, MRP3, MRP4, MRP5, and BCRP.

The foregoing embodiments conducted in accordance with the present disclosure establish that the compounds of Table 1 with the pharmacophore of dibenzocyclooctadiene have activities to effectively reverse MDR cancer, inhibit ABC drug transporters associated with MDR, enhance the anticancer activities of anticancer agents, and directly kill cancer cells. In addition, the compounds of Table 1 have potential in improving oral bioavailability of many diverse drugs that are substrates of ABC drug transporters. It has further been established that the compounds of Table 1 have potential for the pharmacotherapeutic optimization of substrate drugs for ATP drug transporters. Although the potency of the compounds varies with some stronger than others, the class of compounds of Table 1 has the capability to treat cancer and increase the efficacy of anticancer agents as set forth in the claims of the present disclosure. It is further contemplated that chemical modifications may be performed on the chemicals within the class of known compounds of Table 1 or on the pharmacophore of dibenzocyclooctadiene as shown in FIG. 1 to obtain more potent compounds for purposes of the claims in the present disclosure.

Having now described the disclosure in accordance with the requirements of the patent statutes, those skilled in this art will understand how to make changes and modifications in the present disclosure to meet their specific requirements or conditions. Such changes and modifications may be made without departing from the scope and spirit of the disclosure as set forth in the following claims.

1. A method of application of compounds having a pharmacophore of dibenzocyclooctadiene in reversing multidrug resistant cancer, comprising:
preparing a medication comprising at least one of the compounds selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J, Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

2. The method of claim 1, wherein preparing the medication further comprises:
incorporating at least one anticancer chemotherapeutic agent and a pharmaceutically accepted carrier.

3. The method of claim 2, wherein at least one chemotherapeutic agent is selected from the group consisting of:
doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

4. The method of claim 1, wherein preparing the medication further comprises:
formulating the medication for administration in the form of a capsule, caplet, tablet, pill, suspension or liquid.

5. The method of claim 1, wherein preparing the medication further comprises:
incorporating at least one multidrug resistant reversal agent.

6. The method of claim 1, wherein preparing the medication further comprises:
increasing intracellular accumulation of the anticancer agent in multidrug resistant cancer cells.

7. The method of claim 1, wherein preparing the medication further comprises:
inhibiting drug-pump activity of at least one ABC drug transporter.

8. The method of claim 7, wherein at least one ABC drug transporter comprises P-gp, MRP1, MRP2, MRP3, MRP4, MRP5, and BCRP.

9. The method of claim 1, wherein preparing the medication further comprises:
enhancing apoptosis of cancer cells induced by an anticancer agent.

10. The method of claim 1, wherein preparing the medication further comprises:
killing cancer cells

11. A method of increasing efficacy of an anticancer agent comprising:
co-administering to a subject suffering from a multidrug resistant cancer:
(a) a dose of the anticancer agent, wherein the anticancer agent is a substrate of an ABC drug transporter; and
(b) a dose of a compound selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J. Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

12. The method of claim 11, wherein co-administering to a subject suffering from a multidrug resistant cancer further comprises:
administering an optional dose of physiologically acceptable adjuvants, diluents, excipients, or carries.

13. The method of claim 11, wherein the anticancer agent is selected from the group consisting of:
doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

14. The method of claim 11, wherein the dose of the anticancer agent is a therapeutic or subtherapeutic dose.

15. The method of claim 11, wherein the dose of the compound or the analog thereof further comprises:
reducing efflux of the anticancer agent from a cancer cell.

16. The method of claim 11, wherein the dose of the compound or the analog thereof further comprises:
increasing intracellular concentration of the anticancer agent in a cancer cell.

17. The method of claim 11, wherein the dose of the compound or the analog thereof further comprises:
inhibiting a host drug transporter.

18. The method of claim 11, wherein the compound is isolated from a natural source or a chemical synthesis.

19. The method of claim 11, wherein the subject is a mammal.

20. The method of claim 11, wherein the subject is a human.

21. A method of increasing efficacy of an anticancer agent comprising:
co-administering to a subject suffering from a multidrug resistant cancer:
(a) a dose of the anticancer agent, wherein the anticancer agent is a substrate of P-glycoprotein; and
(b) a dose of a compound selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J, Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

22. The method of claim 21, wherein co-administering to a subject suffering from a multidrug resistant cancer further comprises:
administering an optional dose of physiologically acceptable adjuvants, diluents, excipients, or carries.

23. The method of claim 21, wherein the anticancer agent is selected from the group consisting of:
doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

24. The method of claim 21, wherein the dose of the anticancer agent is a therapeutic or subtherapeutic dose.

25. The method of claim 21, wherein the dose of the compound or the analog thereof further comprises:
reducing efflux of the anticancer agent from a cancer cell.

26. The method of claim 21, wherein the dose of the compound or the analog thereof further comprises:
increasing intracellular concentration of the anticancer agent in a cancer cell.

27. The method of claim 21, wherein the dose of the compound or the analog thereof further comprises:
inhibiting P-glycoprotein.

28. A method of increasing efficacy of an anticancer agent comprising:
co-administering to a subject suffering from a multidrug resistant cancer:
(a) a dose of the anticancer agent, wherein the anticancer agent is a substrate of MRP1; and
(b) a dose of a compound selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J, Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

29. The method of claim 28, wherein co-administering to a subject suffering from a multidrug resistant cancer further comprises:
administering an optional dose of physiologically acceptable adjuvants, diluents, excipients, or carries.

30. The method of claim 28, wherein the anticancer agent is selected from the group consisting of:
doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

31. The method of claim 28, wherein the dose of the anticancer agent is a therapeutic or subtherapeutic dose.

32. The method of claim 28, wherein the dose of the compound or the analog thereof further comprises:
reducing efflux of the anticancer agent from a cancer cell.

33. The method of claim 28, wherein the dose of the compound or the analog thereof further comprises:
increasing intracellular concentration of the anticancer agent in a cancer cell.

34. The method of claim 28, wherein the dose of the compound or the analog thereof further comprises:
inhibiting MRP1.

35. A method of increasing efficacy of an anticancer agent comprising:
co-administering to a subject suffering from a multidrug resistant cancer:
(a) a dose of the anticancer agent, wherein the anticancer agent is a substrate of BCRP; and
(b) a dose of a compound selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J, Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

36. The method of claim 35, wherein co-administering to a subject suffering from a multidrug resistant cancer further comprises:
administering an optional dose of physiologically acceptable adjuvants, diluents, excipients, or carries.

37. The method of claim 35, wherein the anticancer agent is selected from the group consisting of:
doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, hydroxyurea, idarubicin, ifosfamide, irinotecan, lomustine, melphalan, mercaptopurine, methotrexate, mitomycin, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

38. The method of claim 35, wherein the dose of the anticancer agent is a therapeutic or subtherapeutic dose.

39. The method of claim 35, wherein the dose of the compound or the analog thereof further comprises:
reducing efflux of the anticancer agent from a cancer cell.

40. The method of claim 35, wherein the dose of the compound or the analog thereof further comprises:
increasing intracellular concentration of the anticancer agent in a cancer cell.

41. The method of claim 35, wherein the dose of the compound or the analog thereof further comprises:
inhibiting BCRP.

42. A method of decreasing toxicity associated with treating a subject with an anticancer agent comprising:
co-administering to the subject having a cancer:
(a) a dose of the anticancer agent; and
(b) a dose of a compound selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J, Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

43. The method of claim 42, wherein co-administering to a patient having a cancer further comprises:
administering an optional dose of physiologically acceptable adjuvants, diluents, excipients, or carries.

44. The method of claim 42, wherein the anticancer agent is selected from the group consisting of:
doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

45. The method of claim 42, wherein the dose of the anticancer agent is a therapeutic or subtherapeutic dose.

46. The method of claim 42, wherein the dose of the compound or the analog thereof further comprises:
reducing efflux of the anticancer agent from a cancer cell.

47. The method of claim 42, wherein the dose of the compound or the analog thereof further comprises:
increasing intracellular concentration of the anticancer agent in a cancer cell.

48. The method of claim 42, wherein the dose of the compound or the analog thereof further comprises:
inhibiting a host drug transporter.

49. A method of enhancing the anticancer activity of an anticancer agent against a cancer cell comprising:
co-administering to a subject suffering from a multidrug resistant cancer:
(a) a dose of the anticancer agent; and
(b) a dose of a compound selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J, Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

50. The method of claim 49, wherein co-administering to a subject suffering from a multidrug resistant cancer further comprises:
administering an optional dose of physiologically acceptable adjuvants, diluents, excipients, or carries.

51. The method of claim 49, wherein the anticancer agent is selected from the group consisting of:
doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, hydroxyurea, idarubicin, ifosfamide, irinotecan, lomustine, melphalan, mercaptopurine, methotrexate, mitomycin, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

52. The method of claim 49, wherein the dose of the anticancer agent is a therapeutic or subtherapeutic dose.

53. The method of claim 49, wherein the dose of the compound or the analog thereof further comprises:
increasing activities of the anticancer agent against cancer.

54. A method of increasing efficacy of an anticancer agent comprising:
co-administering to a subject suffering from a multidrug resistant cancer:
(a) a dose of the anticancer agent, wherein the anticancer agent is a substrate of an ABC drug transporter; and
(b) a dose of ethanol extracts of Schisandra chinensis (Turcz.) Baill fruit.

55. The method of claim 54, wherein the anticancer agent is selected from the group consisting of:
doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin, epirubicin, etoposide, fludarbine, fluorouracil, gemcitabine, herceptin, homoharringtonin, homoharringtonin, hydroxyurea, idarubicin, ifosfamide, irinotecan, lomustine, melphalan, mercaptopurine, methotrexate, mitomycin, mitoxantron, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

56. The method of claim 54, wherein the dose of the anticancer agent is a therapeutic or subtherapeutic dose.

57. The method of claim 54, wherein the dose of the ethanol extracts of Schisandra chinensis (Turcz.) Baill fruit further comprises:
reducing efflux of the anticancer agent from a cancer cell.

58. The method of claim 54, wherein the dose of the ethanol extracts of Schisandra chinensis (Turcz.) Baill fruit further comprises:
increasing intracellular concentration of the anticancer agent in a cancer cell.

59. The method of claim 54, wherein the dose of the ethanol extracts of Schisandra chinensis (Turcz.) Baill fruit further comprises:
inhibiting a host drug transporter.

60. A method of treating a subject suffering from a cancer comprising:
administering a therapeutic dose of a compound selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J, Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

61. The method of claim 60, wherein administering a therapeutic dose of a compound further comprises:
administering an optional dose of physiologically acceptable adjuvants, diluents, excipients, or carries.

62. The method of claim 60, wherein administering the therapeutic dose of the compound further comprises:
killing cancer cells.

63. The method of claim 60, wherein the compound comprises Schisandrin B.

64. A method of increasing oral bioavailability of a drug comprising:
co-administering to a subject:
(a) a dose of the drug, wherein the drug is a substrate of an ABC drug transporter; and
(b) a dose of a compound selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J. Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

65. The method of claim 64, wherein co-administering to a subject further comprises:
administering an optional dose of physiologically acceptable adjuvants, diluents, excipients, or carries.

66. The method of claim 64, wherein the dose of the compound or the analog thereof further comprises:
increasing absorption of the drug by gastrointestinal tract.

67. The method of claim 64, wherein the dose of the compound or the analog thereof further comprises:
inhibiting a host drug transporter.

68. The method of claim 67, wherein the host drug transporter is at least one of P-gp, MRP1, MRP2, MRP4, MRP5 or BCRP.

69. The method of claim 64, wherein the compound is isolated from a natural source or a chemical synthesis.

70. The method of claim 64, wherein the subject is a mammal.

71. The method of claim 64, wherein the subject is a human.

72. A method of optimizing pharmacotherapy of a drug comprising:
co-administering to a subject:
(a) a dose of the drug, wherein the drug is a substrate of an ABC drug transporter; and
(b) a dose of a compound selected from the group consisting of:
Schisandrin A, Schisandrin B, Schisandrol A, Schisantherin A, Schisandrin C, Schisantherin B, Schisantherin C, Schisantherin D, Schisantherin E, Schisantherin F, Schisantherin G, Schisantherin H, Schisantherin I, Schisantherin J, Schisantherin K, Schisantherin L, Schisantherin M, Schisantherin N, Schisantherin O, Schisandrol B, Schisantherinol B, Schisandrol D, Schisantherinol D, Schisandrol E, Schisantherinol E, Methylschisandrol E, Methylschisantherinol E, Angelogomisin P, Tiglogomisin P, Gomisin A, Gomisin B, Gomisin C, Gomisin D, Gomisin E, Gomisin F, Gomisin G, Gomisin H, Angeloylgomisin H, Tigloylgomisin H, Benzoylgomisin H, Gomisin J, Gomisin K1, Gomisin K2, Gomisin K3, Gomisin M1, Gomisin M2, Angeloylgomisin M1, Angeloylgomisin R, Gomisin N, Gomisin O, Epigomisin O, Gomisin Q, Gomisin R, Angelogomisin O, Angeloisogomisin O, Benzoylgomisin O, Benzoylgomisin P, Benzoylgomisin Q, Benzoylisogomisin O, isovaleroyl oxokadsurane, propoxyl oxokadsurane, acetyoxyl oxokadsurane, benzoyl oxokadsurane, isovaleryol oxokadsuranol, acetylbinankadsurin A, angeloylbinankadsurin A, caproyl-binankadsurin A, kadsurin, kadsurarin, kadasutherin, isokadsuranin, neoisostegane, neokadsuranin, interiorin, [5,8-epoxy-6,7-dimethyl 2′,3′,2″,3″-dimethylenedioxy-4′,1″-dimethoxy-1,2:3,4-dibenzo-1,3-cyclooctadiene], binankadsurin A, benzoylbinankadsurine A, isovaleroylbinankadsurin A, angeloylbinankadsurin A, and isobutyroylbinankadsurin A; or an optical isomer, diasteromer, enantiomer, pharmaceutically-accepted salt, or analog thereof.

73. The method of claim 72, wherein co-administering to a subject further comprises:
administering an optional dose of physiologically acceptable adjuvants, diluents, excipients, or carries.

74. The method of claim 72, wherein co-administering to a subject further comprises:
modulating the permeability of at least one of the blood-brain barrier, blood-testis barrier, blood-nerve barrier, or the fetal-maternal barrier to the drug; or modulating at least one of the hepatobiliary or renal excretion of the drug.

75. The method of claim 72, wherein co-administering to a subject further comprises:
inhibiting a host drug transporter.

76. The method of claim 75, wherein the host drug transporter is at least one of P-gp, MRP1, MRP2, MRP3, MRP4, MRP5, or BCRP.

77. The method of claim 75, wherein the compound is isolated from a natural source or a chemical synthesis.

78. The method of claim 72, wherein the subject is a mammal.

79. The method of claim 72, wherein the subject is a human.
